(19) United States
(12) Patent Application Publication
(10) Pub. No.: US 2011/0245744 A1
(43) Pub. Date: Oct. 6, 2011

(54) FLEXIBLE MEDICAL BARRIER SYSTEMS AND METHODS OF USE

(75) Inventors: Alexander Harrison Bellamy, Bothell, WA (US); Richard Radford, Auburn, WA (US)

(73) Assignee: CENORIN, LLC, Kent, WA (US)

(21) Appl. No.: 13/079,291

(22) Filed: Apr. 4, 2011

Related U.S. Application Data
(60) Provisional application No. 61/320,886, filed on Apr. 5, 2010.

(51) Int. Cl. A61F 15/00

(52) U.S. Cl. 602/3

(57) ABSTRACT

The present application relates generally to a medical barrier, and in particular, to a medical barrier comprising a sheet through which a patient's appendage can be inserted, a flexible band connected to the sheet, and a stabilization tab to stabilize the sheet during the application of the medical barrier to the patient.

### CROSS REFERENCE TO RELATED APPLICATIONS

This application claims priority to U.S. Provisional Patent Application No. 61/320,886, filed Apr. 5, 2010, entitled FLEXIBLE MEDICAL BARRIER SYSTEMS AND METHODS OF USE, which is hereby incorporated herein by reference in its entirety.

### BACKGROUND OF THE INVENTION

1. Field of the Invention

The present disclosure relates generally to medical barriers. Certain embodiments relate to medical barrier devices. Certain embodiments relate to methods of manufacturing medical barriers. Certain embodiments relate to methods of applying a medical barrier to a patient.

2. Description of the Related Art

Many types of medical barriers for protecting a portion of a patient's body, such as an appendage, from exterior environmental elements are known in the art. However, it is difficult to design a simple medical barrier that can be easily applied to and removed from the patient while still providing adequate and reliable coverage to the portion of the patient needing protection.

A number of medical barriers with various designs have been developed. Some of these conventional medical barriers have been designed with complex and often expensive structures, such as zippers or other elaborate fastening systems to secure the barrier to the patient. Simpler designs include straps affixed to the barrier that do not adequately or reliably seal the protected portion of the patient's body from external elements.

### SUMMARY

One embodiment provides a medical barrier comprising a sheet. The sheet comprises an edge circumscribing an opening disposed at a first end of the sheet, through which a portion of a patient's appendage can be inserted so as to be at least partially surrounded by the sheet. The medical barrier comprises a flexible band connected to the sheet. The band comprises a substrate, an adhesive layer, and a release layer disposed on the adhesive layer so that the adhesive layer is between the substrate and the release layer. The release layer is peelable from the adhesive layer to expose the adhesive layer. A portion of an end of the band comprises a stabilization tab that includes portions of the substrate, adhesive layer, and release layer. The stabilization tab is predisposed to be severable from a remainder of the band.

Another embodiment provides a medical barrier comprising a sheet. The sheet comprises an edge circumscribing an opening disposed at an end of the sheet, through which a portion of a patient's appendage can be inserted so as to be at least partially surrounded by the sheet. The medical barrier comprises a flexible band connected to the sheet. The band comprises a substrate, an adhesive layer, and a release layer disposed on the adhesive layer so that the adhesive layer is between the substrate and the release layer. At least a main portion of the release layer is peelable from the adhesive layer to expose the adhesive layer. The release layer comprises the main portion and a substantially smaller handling portion, the handling portion being at or near a free end of the band. The main portion and the handling portion are severed or predisposed to be severable with respect to each other, such that the main portion of the release layer can be removed from the band without removing the handling portion of the release layer.

Another embodiment provides a medical barrier comprising a sheet. The sheet comprises an edge circumscribing an opening disposed at an end of the sheet, through which a portion of a patient's appendage can be inserted so as to be at least partially surrounded by the sheet. The medical barrier comprises a flexible band connected to the sheet. The band comprises a substrate, an adhesive layer and a release layer disposed on the adhesive layer so that the adhesive layer is between the substrate and the release layer. The release layer is peelable from the adhesive layer to expose the adhesive layer. The substrate is attached to the sheet at or near a proximal end of the substrate. A proximal end portion of the release layer extends proximally beyond the proximal end of the substrate and beyond a proximal end of the adhesive layer. The proximal end portion of the release layer is detached from the sheet.

Another embodiment provides a medical barrier comprising a sheet. The sheet comprises an edge circumscribing an opening disposed at an end of the sheet, through which a portion of a patient's appendage can be inserted so that the edge surrounds a perimeter of the appendage. The medical barrier comprises a flexible band connected to the sheet. The band comprises a substrate, an adhesive layer, and a release layer having an inner surface disposed on the adhesive layer, so that the adhesive layer is between the substrate and the release layer. The release layer is peelable from the adhesive layer to expose the adhesive layer. An end segment of the band is attached to the sheet such that a portion of the release layer extending along the end segment of the band has an outer surface that faces away from the sheet. The end segment of the band is oriented substantially along a circumference of the sheet's edge and positioned substantially at said edge.

Another embodiment provides a medical barrier comprising a sheet. The sheet comprises an edge circumscribing an opening disposed at an end of the sheet, through which a portion of a patient's appendage can be inserted so as to be at least partially surrounded by the sheet. The medical barrier comprises a flexible band comprising a substrate, an adhesive layer having an inner surface disposed on the substrate, and a release layer having an inner surface disposed on an outer surface of the adhesive layer. The adhesive layer is between the substrate and the release layer, the release layer is peelable from the adhesive layer to expose the outer surface of the adhesive layer. A segment of the band is attached to an outer surface of the end of the sheet such that an outer surface of the release layer at the band's attached segment faces away from the sheet. The attached segment of the band is oriented generally along a circumference of the edge, an overhang portion of a width of the attached segment of the band extending beyond the edge. A removable stabilization tab portion of the substrate or the release layer within the overhang portion of the band is severed or predisposed to be severed from a remainder of the substrate or release layer and is peelable from the adhesive layer to expose a stabilization tab portion of the adhesive layer, the stabilization tab portion of the adhesive layer configured to adhere to a patient's appendage when inserted through the opening.

Another embodiment provides a medical barrier comprising a sheet. The sheet comprises an edge circumscribing an opening disposed at an end of the sheet, through which a portion of a patient's appendage can be inserted so as to be at least partially surrounded by the sheet. The medical barrier comprises an elastic band connected to the sheet, a plurality of adhesive regions disposed on and spaced along a length of one side of the band, each adhesive region being covered by a peelable tab. The medical barrier comprises a first adhesive strip connecting a proximal end portion of the band to the sheet, and a second adhesive strip connecting the proximal end portion of the band to the sheet.

Other embodiments provide various methods of using the aforementioned medical barrier embodiments.

For purposes of summarizing the invention and the advantages achieved over the prior art, certain objects and advantages of the invention have been described above and as further described below. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

The appended drawings are schematic, not necessarily drawn to scale, and are meant to illustrate and not to limit embodiments of the invention.

FIG. 1 shows an embodiment of a medical barrier applied to a user.

FIG. 2 shows a side-perspective view of another embodiment of a medical barrier.

FIG. 3A shows a side-perspective view of another embodiment of a medical barrier.

FIG. 3B shows a side-perspective view of the medical barrier shown in FIG. 3A, with a stabilization tab severed therefrom.

FIG. 4A shows a partial side-perspective view of an embodiment of the medical barrier shown in FIGS. 3A and 3B, with the stabilization tab partially removed therefrom by a user.

FIG. 4B shows a close-up view of an embodiment of the stabilization tab shown in FIGS. 3A and 3B.

FIGS. 5A-5E show a method of using an embodiment of a medical barrier.

FIG. 6A shows a side perspective view of another embodiment of a medical barrier.

FIGS. 6B-6F show a method of using the medical barrier of FIG. 6A.

FIG. 7A shows a partial side perspective and partially exploded view of an embodiment of a medical barrier.

FIG. 7B shows a partial side perspective and partially exploded view of an embodiment of a medical barrier.

FIG. 8A shows a side perspective view of another embodiment of a medical barrier that has an elastic band assembly.

FIG. 8B shows a side perspective view of another embodiment of a medical barrier that has an elastic band assembly with multiple attachment points.

FIGS. 8C-8E show close-up, partial side views of various embodiments of the elastic band assemblies shown in FIGS. 8A and 8B.

### DETAILED DESCRIPTION OF THE DRAWINGS

The present inventions relate to medical protective barriers. Such barriers prevent foreign contaminants, such as liquids, from entering a protected area on a patient covered by, for example, a medical bandage, or an area having an exposed, open wound. Such barriers may be leak-proof and/or leak-resistant, to prevent moisture contamination of the protected area while a patient is showering or exposed to outdoor precipitation. Many conventional barriers include complex and expensive systems to provide sufficient sealing protection between the patient and the exterior environment, and to adequately hold the barrier in place, both during and after application to the patient. Other conventional barriers are difficult to apply to, or remove from, a patient's appendage, particularly for a patient with only a single hand available. Some conventional barriers may slip and rotate relative to the appendage during or after application, increasing the likelihood of contamination of the protected area through or around the barrier. Other conventional barriers may be limited to a certain size of appendage on which they may efficiently be applied; for example, when a larger conventional barrier is wrapped around a smaller appendage, excess material may form a series of pleats or folds around the appendage, forming leakage channels into the protected area. The embodiments of the medical barriers described further herein prevent contamination, such as wetting, of the protected area using a minimal amount of materials arranged in an efficient, reliable, and easy-to-use manner, with the flexibility of being capable of use on various sized appendages.

Some embodiments of the present application include a water-resistant medical barrier having a flexible sheet, or in some embodiments, a tube, with two sides, an inside and an outside. In some embodiments, the medical barrier can be shaped like a sock, sleeve or glove that fits over a user's appendage. In some embodiments, adhesive is coupled to at least one of the sides of the sheet to attach the medical barrier to a user.

The medical barrier can include at least one band. The band can be stretchable or substantially non-stretchable. The band can be elastic or non elastic. The band can have an adhesive layer or sealing strip over at least a part of the band. The band can be used to wrap around an open edge of the medical barrier and secure and seal the medical barrier around a user's appendage. The band is preferably configured to substantially circumscribe a circumference of an edge of at least one opening of the medical barrier and adhere or seal through pressure the edge from moisture and the surrounding environment. A further aspect of the present application is the strategic placement of the band on the barrier. This placement on the barrier preferably assures a complete seal around the user's appendage, preventing ingress of fluids or other contaminants into the protected area.

A further aspect of the present application is one or more user-friendly stabilizing members, such as tabs or adhesive pads, provided on the inside and/or outside of the barrier that allow a single user to apply the medical barrier with one hand. The stabilizing member holds the barrier in place while the user wraps the band around an appendage, preventing rotation of the barrier as the user applies tangential force while wrapping the sealing strip around an open edge of the protective barrier.

In some embodiments, the stabilizing member comprises a tab attached to a flexible band at a perforated line, notch, or other characteristic that predisposes the stabilizing member to be severable from the band in a predetermined manner. The user can remove the stabilizing member prior to securing the medical barrier to the user's body and secure a portion of the medical barrier so that the barrier folds as desired and does not twist or move as the user applies the band. These aspects are particularly advantageous because if the barrier were to bunch up or fold in a problematic location along the seal between the medical barrier and the user's body, a channel may form that allows moisture to flow into the protected area, especially when a user moves the related appendage.

These aspects provide the further benefit that the medical barrier is easily adaptable for use on small or large appendages, and the anti-channeling property described above is achieved on small and large appendages alike These and other advantages are achieved with the present embodiments.

Description will now be made of embodiments of a medical barrier. It should be understood that the disclosed embodiments present examples of the present inventions for illustrative purposes, and that the scope of the present inventions is not limited to the embodiments disclosed herein.

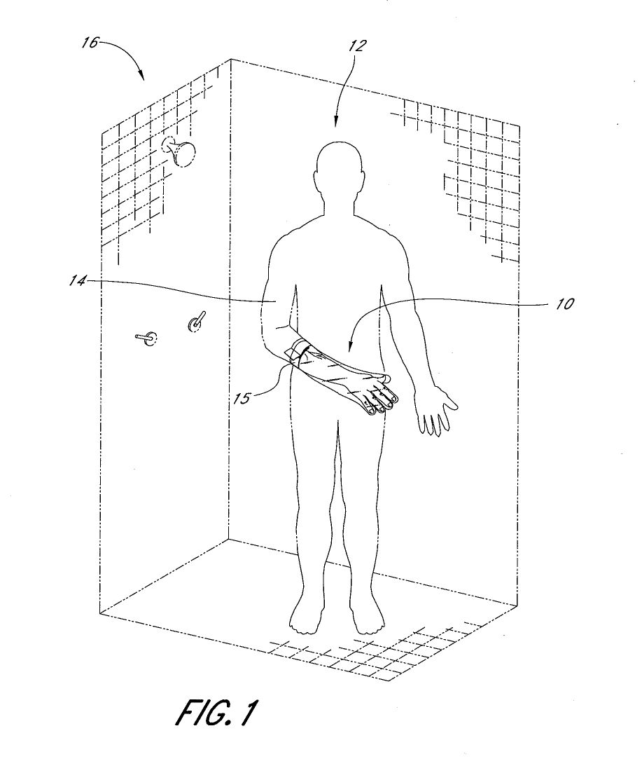

FIG. 1 shows an embodiment of a medical barrier 10 applied to a user or patient 12 on an appendage 14 using a flexible band 15 in an exemplary potential environment of use, such as a shower 16. It will be understood that although the embodiments of the medical barriers described herein are shown on humanoid patients, “patient” or “user” as used herein are not limited to any particular species of animal, and the embodiments of the medical barrier described herein can be used on non-human animals. Additionally, “patient” can refer to any size or type of animal, living or dead, with an appendage onto which the medical barrier may be applied. Further, “appendage” as used herein can refer to any body member extending from the axial trunk of the patient, e.g., an arm, leg, finger, foot, or hand, or any portion thereof.

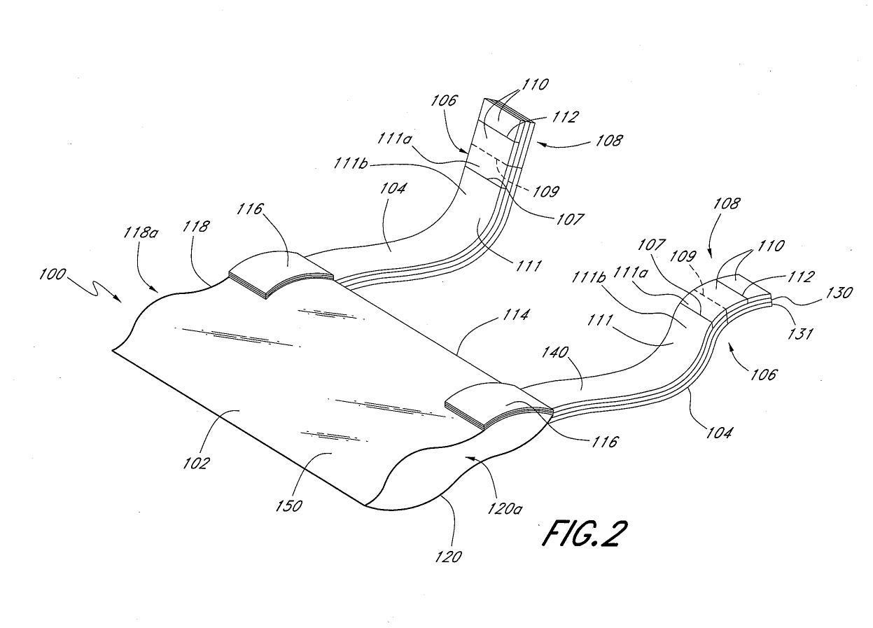

FIG. 2 illustrates an embodiment of a medical barrier 100 that can be applied to a patient's appendage (not shown). The medical barrier 100 can comprise a sheet or film 102 and one or more flexible strips or bands 104 attached thereto for holding the sheet 102 to a patient. Sheet 102 can be any of many different shapes and materials. In an embodiment, the sheet 102 can comprise a sheet of material that can partially conform to and partially surround a portion of a patient's appendage to form a substantially curved shape. The sheet 102 can form a generally frustro-conical, tube-like or cylindrical shape, with an opening formed at either or both of its opposed ends, and a channel or cavity therein into which a portion of a patient's appendage can be inserted. In the embodiment shown in FIG. 2, the sheet 102 can comprise a substantially cylindrical shape with openings 118a and 120a circumscribed at each of its opposed ends by edges 118 and 120, respectively. In other embodiments, such as those shown in FIGS. 1, 3A-3B, 6A, 6C, 6E-6F, and 7A-8B, the sheet can comprise a bag-like, tube-like or cylindrical shape with an opening circumscribed by an edge at an open end to receive a patient's appendage, and an opposed closed end. In other embodiments, the closed end of sheet 102 can comprise the shape of an extremity to an appendage, such as a hand, paw, or foot. In some embodiments, a first sheet having a tubular end (such as the configuration shown in FIG. 2), can be attached to a separate sheet shaped like an extremity of an appendage (e.g., to provide an arm-length glove), using any of many attachment methods known in the art, e.g., adhesive, ultrasonic or thermal bonding, hook/loop fasteners, and the like.

The sheet 102 can comprise any of many different types of films, membranes, or substrates. The sheet 102 may be any of myriad compositions suitable for short-term or long-term contact with a user's skin. The sheet 102 can comprise a waterproof or water-resistant material. In some embodiments, any portion of the sheet 102 can be coated, for example, with pharmaceutical or other therapeutical treatments to improve the comfort and health of the user. In some embodiments, the sheet 102 may comprise a thin, flexible, and in some embodiments, elastic, stretchable film. The sheet 102 may comprise one or more layers of material, and may comprise one or more materials along its length or width. The sheet 102 may alternatively or additionally be comprised of a hydrophobic or hydrophilic material, including, but not limited to, polymers and plastics and water-vapor-breathable films, layers, and materials. The sheet or film 102 may be any color, clear or opaque to any degree, and may be printed, for example, with brand or source identification, constructions or application, aesthetic decorations, and the like. The sheet 102 may further be shaped in various ways to carry out the purposes of treatment, including, without limitation, rectangular, square, ovular, circular, or any other suitable regular or irregular shape. For example, the sheet or film 102 may further comprise pocket portions capable of holding items such as jewelry or medical implements while the user is, for example, showering. In a preferred embodiment, the sheet 102 is a thin, waterproof or water-resistant film that is sufficiently flexible to be at least partially wrapped around a portion of a patient's appendage and held in place with the flexible band 104.

The flexible band 104 of the barrier 100 can comprise a flexible, and in some embodiments, stretchable or elastic strip, band, or strap with a base end (or proximal end) connected to the sheet 102, and a free end movable relative to the base end. In some embodiments, the length of the band 104 can be greater than or equal to the perimeter (e.g., circumference) of the open edge 118, 120 of the medical barrier that can be sealed against the patient's skin as discussed further herein. The flexible band 104 can comprise a substrate 131, an adhesive layer 130 (or in some embodiments, layers), and a release liner, or release layer 111. The release layer 111 can be disposed on the adhesive layer 130 so that the adhesive layer 130 is between the substrate 131 and the release layer 111. As used herein, the term “circumference” does not necessarily imply circularity, unless so indicated.

The substrate 131 can comprise any of the material or materials described herein for sheet 102, and can comprise the same or different material as sheet 102. Substrate 131 can comprise a flexible, stretchable, elastic or non elastic, waterproof or water-resistant film. In some preferred embodiments, substrate 131 comprises a material with sufficient flexibility to partially wrap flexible band 104 around a user's appendage, and with sufficient strength to hold and seal sheet 102 to the user, and to support the adhesive layer 130 during use of sheet 102, as described further herein.

The adhesive layer 130 may comprise, for example, without limitation, pressure-sensitive, thermo-sensitive, and/or radiation-sensitive adhesive materials. The adhesive layer 130 can comprise non-toxic adhesives that allow waterproof adhesion and comfortable removal from contact with a patient's body either alone or in combination with other aspects of the present disclosure. The adhesive layer 130 can comprise a continuous layer of adhesive, or in some embodiments, a plurality of intermittent or selectively located adhesive regions. In addition to the various compositions possible within the examples described elsewhere in the present disclosure, the adhesive used within the adhesive layer portions described herein can be disposed using a variety of methods in a variety of fashions and in a variety of configurations or patterns. Some embodiments may optimize the skin contact and adhesive properties of the medical protective barrier. In this manner, the protected area is shielded from water, soap and the like when cleansing. In other embodiments, the adhesive circumscribes the protected area on the patient with the sheet covering the protected area (e.g., the intersection between an introducer port for a PICC line and patient's skin). The adhesive may be placed on other portions of the medical protective barrier besides the areas or portions described above. Such alternative configurations include, but are not limited to, adhesive applied to areas extending across only the periphery and/or edges of a sealing band or stabilization tab, and areas within the medical protective barrier other than its periphery, such as a line on the inner and/or outer surface of the sheet 102 extending from its proximal end and along its length partially or fully making its way to a distal end of the medical barrier. In some embodiments, a plurality of adhesive layers is applied in a single or variety of patterns. Examples of adhesive patterns that are used in various embodiments include adhesive disposed along a straight line path, in a circular pattern, dot pattern, or a zigzag pattern on a portion of the medical barrier. In other embodiments, the adhesive layer is disposed randomly. In some embodiments, adhesive is applied only at a portion of the flexible band that will overlap a fold. This portion can be determined by measurement and by directed placement that takes advantage of the predictable folds that form due to the stabilization tab, which are described elsewhere herein. This saves cost on adhesive materials and minimizes risk of discomfort in removing the adhesive while ensuring that adhesive is positioned to cover the fold portion, a part of leak-resistant medical barriers typically prone to channeling and leakage. In such embodiments, the band seals the sheet against the user even in regions with no adhesive by way of the pressure seal created by wrapping the band around the user's appendage. While regions with adhesive can beneficially be sealed by both a pressure seal and an adhesive barrier seal, a purely pressure seal can provide adequate protection when used in conjunction with the other disclosed aspects of the inventions.

The release layer 111, adhesive layer 130, and substrate 131 can comprise one or more materials with various adhesive properties (e.g., coefficient of friction, surface tension, surface smoothness or roughness, and the like), to allow either, neither or both of release layer 111 and/or substrate 131 to be peelable or removable from adhesive layer 130, without peeling or removing the other of release layer 111 or substrate 131. In an embodiment, the release layer 111 can comprise a protective film made from one or more materials with adhesive properties that allow release layer 111 to be peelable, or removable, from adhesive layer 130, to expose adhesive layer 130, without removing substrate 131 from adhesive layer 130. The level of adhesion between the adhesive layer 130 and the substrate 131 and/or between adhesive layer 130 and the release layer 111 can be adjusted by coating release layer 111 and/or substrate 131 with a coating.

The flexible band 104 of the medical barrier 100 can be attached to a side, or as illustrated in FIG. 2, an outer surface 150 of sheet 102 at an attachment portion 116, or base, of band 104. Attachment portion 116 can be attached to sheet 102 in many different ways known in the art, such as with adhesive, ultrasonic or thermal bonding, mechanical fasteners, and the like. In a preferred embodiment, the flexible band 104 can be attached partially along the edge 118 or 120 of sheet 102, proximate to one of the openings 118a or 120a in sheet 102. In another preferred embodiment, the flexible band 104 can be attached to sheet 102 so that the band 104 can partially extend beyond the edge 118 or 120 so as to partially overhang opening 118a or 120a, and overlap the sheet 102 and contact the user's skin during application. Depending on the application, user, or environment of use, these and other configurations can be ideal. Additionally, manufacturing constraints or optimization can provide the parameters for ideal placement of the attachment portion 116 of each flexible band 104 to sheet 102.

In some embodiments, the length of the perimeter (i.e., the lineal length or circumference), i.e., of edges 118 and 120 of sheet 102, may exceed the user's appendage perimeter in the area of coverage. This excess length in the edges of 118 and 120 may allow the appendage to be more easily inserted into the openings 118a and 120a of sheet 102. The placement of the flexible band 104 (on the top side 150 of sheet 102) and the stabilization tab 108 described further herein can allow the entire perimeter of the open ends 118a and 120a to be covered by a portion of adhesive layer 130 of the adhesive wrap section 140, even in a fold or overlap area that may be formed to reduce the effective size of the openings 118a, 120a. Full coverage around the entire perimeter of edges 118 and 120, and/or sealing within the fold or overlap area with the flexible band 104 can assure that fluid will not ingress into the protected area of the barrier 100. Without such coverage and/or sealing, the fold or overlap area of sheet 102 could form unwanted channels or take on an undesired bunched or pleated shape, thus, all edges of sheet 102 would not be covered with adhesive, and ingress of fluids will occur and permit fluid leakage into the protected area.

In some embodiments, the flexible band 104 can comprise a main section 140, and either, neither or both of a handling section 106 and a stabilization tab section 108. In some embodiments, flexible band 104 can comprise one or more of either or both handling sections 106, and one or more stabilization tab sections 108. In some embodiments, the main section 140 includes the attachment portion 116. The main section 140, handling section 106 and stabilization tab section 108 of the flexible band 104 may each comprise portions of each of release layer 111, adhesive layer 130 and substrate 131. In a preferred embodiment, the flexible band comprises the main section 140 (including the attachment portion 116), a handling section 106, and a stabilization tab section 108, collectively comprising a single continuous layer of each of adhesive layer 130, release layer 111, and substrate 131.

The stabilization tab 108 serves to stabilize the medical barrier 100 on the user's appendage while applying the medical barrier 100, as described further herein. In some embodiments, the stabilization tab 108 can be predisposed to be severable from the remainder of flexible band 104. After removing the stabilization tab 108, the user can adhere it to the sheet 120 so that the tab 108 overhangs an edge 118 or 120, and so that the overhanging portion can adhere to the user's skin. The user can optimize the tab's placement to assure that the fold or overlap of the sheet 102 discussed above is controlled as to its location and shape (lays flat and wrinkle free) thereby enhancing the sealing process. This controlled placement has a strategic advantage in preventing ingress of fluids into the protected area and will be discussed in detail below.

The stabilization tab 108 can be predisposed to be severable from band 104 in many different ways. In an embodiment, the stabilization tab section 108 comprises a portion of adhesive layer 130, substrate 131 and release layer 111. Band 104 can comprise a division 109 (illustrated as a straight line) extending across the width of band 104 between the stabilization tab section 108 and the main section 140. Division 109 allows the stabilization tab section 108 to be removed from the remainder of the flexible band 104 (e.g., main section 140) by tearing along the division 109. Division 109 can be formed in any of many different ways known in the art. In a preferred embodiment, division 109 is a perforation line comprising a plurality of aligned perforations extending at least partially across the width of and at least partially through the thickness of band 104. In other embodiments, division 109 can be a heavy crease or a strip of releasable adhesive that bonds main portion section 140 to stabilization tab 108. In some embodiments, the stabilization tab 108 may comprise one or more divisions 109 to form more than one stabilization tab. In the illustrated embodiment of FIG. 2, stabilization tab 108 is positioned proximate to the free end, or distal end, of band 104.

The portion of the release layer 111 on the stabilization tab 108 can comprise two release layer portions 110 with a division 112 therebetween. The two release layer portions 110 can be configured to be separable at division 112 by, e.g., a kiss cut (i.e., a cut that penetrates through only the release layer 111) or by aligned perforations. The two release layer portions 110 can be separable from each other to allow a user to more easily remove them from stabilization tab 108, expose the portion of the adhesive 130 on stabilization tab 108, and adhere stabilization tab 108 to a user and sheet 102 as described further herein.

The handling tab 106 aids in application of barrier 100 to a patient by providing an adhesive-free area for the user to hold while handling flexible band 104. In an embodiment, release layer 111 comprises a smaller handling portion 111a near the distal end of the band 104, and a main portion 111b extending substantially throughout a remainder of the length of the band. The portions 111a and 111b are preferably severed or severable with respect to each other at division 107, through e.g., a kiss cut or perforation through release layer 111. Division 107 allows a user to remove the main portion 111b of release layer 111 from band 104 without removing the handling portion 111a. Thus, a user may grasp the handling portion 111a of handling tab 106 when moving the free end of band 104 to apply barrier 100 to the user's appendage.

With respect to some of the above described embodiments of the medical barrier, an advantage of providing the flexible band 104 with the main section 140, stabilization tab 108 and/or the handling tab 106 each comprising portions of release layer 111, adhesive layer 130, and substrate 131, is that manufacturing costs are reduced. Specifically, the main section 140, stabilization tab 108, and/or the handling tab 106 can be manufactured in fewer steps, by applying and/or attaching a single length of substrate 131, adhesive layer 130, and release layer 111 to form band 104. Including stabilization tab 108 as part of band 104 benefits the user because the stabilization tab 108 is less likely to be inadvertently separated from the remainder of the medical barrier 100 before use. This configuration also encourages the user to actually use the stabilization tab 108. If the stabilization tab 108 were not provided as part of the flexible band 104, the user might decide that the stabilization tab 108 need not be applied, or might forget to apply it or lose the stabilization tab 108. Such circumstances would result in a higher likelihood of a channel forming in the medical barrier 100, which allows for leakage of fluid into the barrier.

Another advantage of providing the stabilization tab 108 severably attached to the flexible band 104 along the division 109 is that rather than requiring a user to use additional tools to create a stabilization tab by, for example, cutting off an end portion of the flexible band 104 with scissors, the stabilization tab 108 can be a pre-defined ideal width and length and can be separated and used without potentially dangerous or inaccessible tools and potentially faulty decision-making by the user (e.g., the user tearing off a tab that is too small or too large. For example, the barrier could be used in a controlled hospital environment where sharp objects are not permitted or advisable. Additionally, users with minimal competency or very little medical experience can successfully apply medical barriers having a band 104 with a stabilization tab as set forth in the present disclosure.

In some embodiments described herein, the flexible bands (e.g., band 104) of the medical barriers (e.g., medical barrier 100) can be provided in a kit separately from the sheet (e.g., sheet 102). In such an embodiment, the flexible band and/or sheet may be provided with an additional adhesive strip, mechanical fastening system, or the like, so that a user may attach an attachment portion of the flexible band (e.g., portion 116) to the sheet. It is well-known that the nature and extent of injuries varies widely. Besides lowering manufacturing costs, a kit configuration provides greater flexibility and choice for end users, such as nurses or hospital patients, and provides a medical barrier preparation system that can accommodate a wide range of variability in, for example, user, and environment, nature and extent of injury. For example, a user having very long appendages may require a medical barrier to cover an area much greater than a typical user, which might be difficult to achieve using barriers with pre-attached bands. Rather than require a manufacturer to produce a larger model of medical barrier for a small subset of users, the kit configuration provides the flexibility that would allow a user to prepare a custom medical barrier. In this example, the user might attach two sheet portions to one another in a serial manner by wrapping a flexible band or other strip optimally designed for this purpose—such as a band that is twice as wide as the flexible band 104 shown—with the adhesive exposed, around the edge portion 118 of one sheet and the edge portion 120 of another sheet. Using this method of preparing a customized medical barrier, the user will have created a medical barrier capable of covering the unusually large area required by the user of this example.

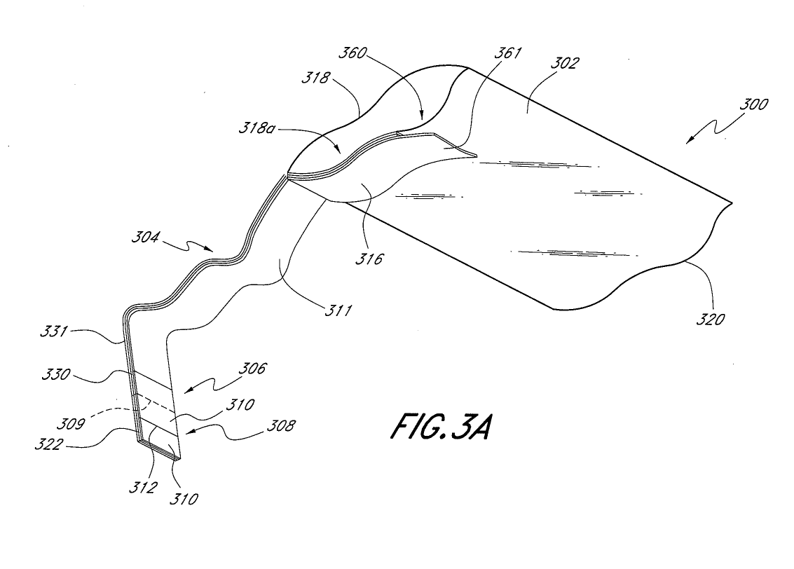

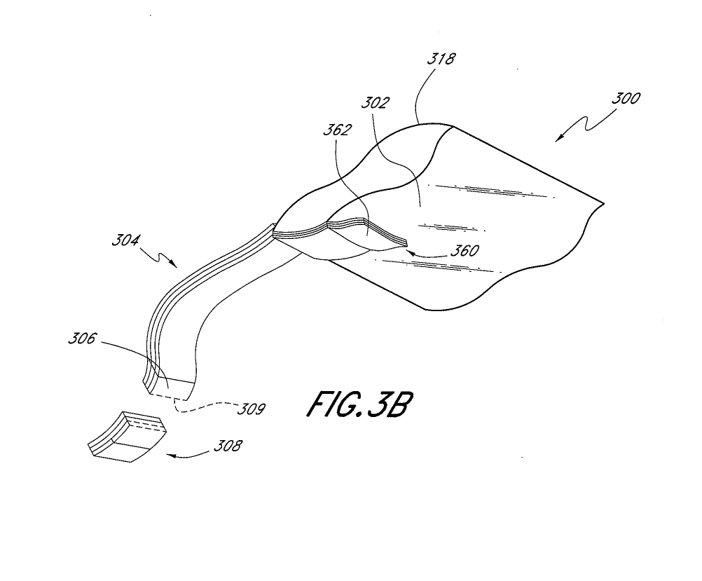

FIG. 3A shows a medical barrier 300 according to another embodiment. Many of the components in the medical barrier 300 are substantially similar to the components of the medical barrier 100 shown in FIG. 2, and function similarly to the manner described herein for medical barrier 100. One difference between the illustrated medical barrier 300 and the medical barrier 100 is that the medical barrier 300 is illustrated, for exemplary purposes only, as comprising a sheet 302 with an edge 318 circumscribing an opening 318a at one end, and a closed end 320 at its opposed end. Sheet 302 is also shown, for exemplary purposes only, with a single strap 304 proximate to the opening 318a. In the exemplary illustrated embodiment, the barrier 300 comprises a bag-shape. Although the top edge 318 is illustrated as smooth around its perimeter in this embodiment, other embodiments can have fully or partially pleated edges that may be lined with an elastic material that tends to bring and keep the edge 318 of the sheet 302 into contact with the user and provides a conformable placement grip from the sheet to the user's appendage that is useful in holding the barrier 300 stable while the user applies the band 304. While the top edge 318 circumscribes an opening in the sheet 302, the sheet 302 converges upon itself and does not circumscribe an opening at its bottom edge 320 in the present embodiment. Thus, a user need only seal the top edge 318 of the medical barrier around an appendage to create a waterproof or water-resistant environment.

The medical barrier 300 can include a band 304 comprising an adhesive layer 330, substrate 331, and release layer 311 that are substantially similar to and that function similarly to the manner of the strap 104, adhesive layer 130, substrate 131, and release layer 111 of the medical barrier 100. The band 304 can comprise an attached portion 316, a stabilization tab 308, a division 309, and a handling portion 306 that are substantially similar to and that function similarly to the manner of the stabilization tab 108, attached portion 116, division 109 and handling portion 106 of the medical barrier 100. A difference between the illustrated medical barrier 300 and the medical barrier 100 is that the strap 304 of the medical barrier 300 can comprise a detached portion 360 positioned at or near the proximal end of the band 304. In the illustrated embodiment, the detached portion 360 is not attached to the sheet 302, and extends beyond, and is removable or peelable from, attached portion 316. The detached portion 360 can comprise any of many different shapes, and can extend beyond attached portion 316 in any of many different ways. In a preferred embodiment, the detached portion 360 extends proximally beyond attached portion 316, and is substantially longitudinally aligned with band 304. In a further preferred embodiment, the detached portion 360 comprises a substantially similar width as band 304. In an embodiment, the detached portion 360 can comprise a proximal end portion of each of the adhesive layer 330, substrate 331, and release layer 311. In such an embodiment, a division, substantially similar to division 309, can be formed between the detached portion 360 and the attached portion 316, so that detached portion 360 can be severed from the attached portion 316. In such an embodiment, the detached portion 360, when severed from the attached portion 316, can be used as a stabilization tab 362 (FIG. 3B), functioning similarly to the stabilization tabs 108 (FIGS. 2) and 308 (FIG. 3A). Referring to FIG. 3A, in a preferred embodiment, detached portion 360 comprises a proximal end portion 361 of release layer 311, and does not comprise a proximal end portion of the adhesive layer 330 and the substrate 331. In this preferred embodiment, the proximal end portion 361 of release layer 311 forms a removal tab that can be grasped by a user to facilitate removal of release layer 311 by pulling end portion 361 away from the attached portion 316 of band 304.

FIG. 3B shows a side-perspective view of an embodiment of the medical barrier 300 shown in FIG. 3A, wherein the stabilization tab 308 has been severed from the free end of band 304.

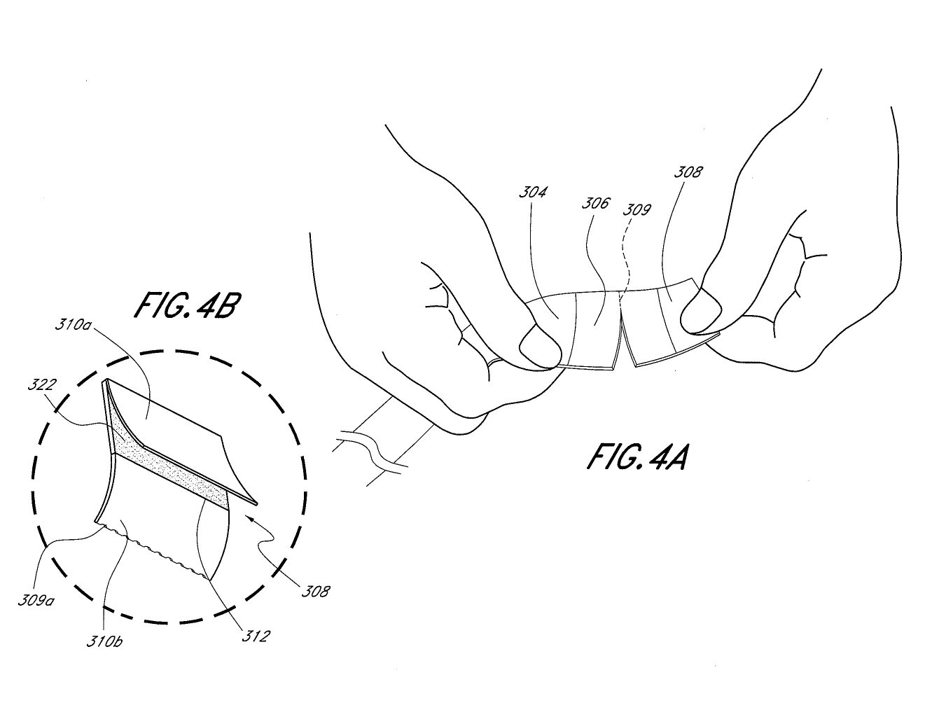

FIG. 4A shows a partial side-perspective view of an embodiment of the medical barrier 300 shown in FIGS. 3A and 3B, with the stabilization tab 308 partially removed from the band 304 by a user tearing along the division 309.

FIG. 4B shows a close up view of an embodiment of the stabilization tab 308 shown in the medical barrier of FIGS. 3A and 3B, after being removed from the band 304. Stabilization tab 308 can comprise two release layer portions 310a, 310b with a division 312 therebetween, which preferably are substantially similar to and function similarly to the release layer portions 110 and division 112 shown in FIG. 2. In the illustrated embodiment, the stabilization portion 308 comprises a serrated edge 309a remaining after separation of the stabilization tab portion 308 along a perforated division 309. The release layer portion 310a can be removed at a division 312 from the tab 308 as shown, revealing a first portion of an adhesive layer 322. During application, the second portion 310b of the release layer may initially be left attached to adhesive layer 322 during the application of the stabilization tab 308, allowing the user to have an adhesive-free area to grasp. After the first portion of the stabilization tab 308 is applied, the second portion of the adhesive layer 310b can be removed, and the remainder of the stabilization tab 308 can be applied. In the illustrated embodiment, the tab is square-shaped and appears smoothly textured, while in other embodiments the tab can be advantageously shaped and/or textured differently, such as a star, letter, crescent, or circle, and the like.

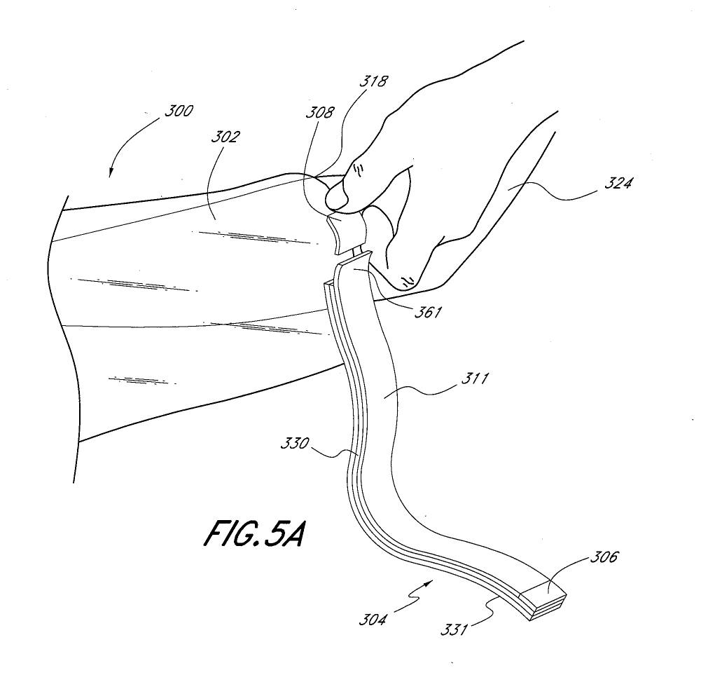

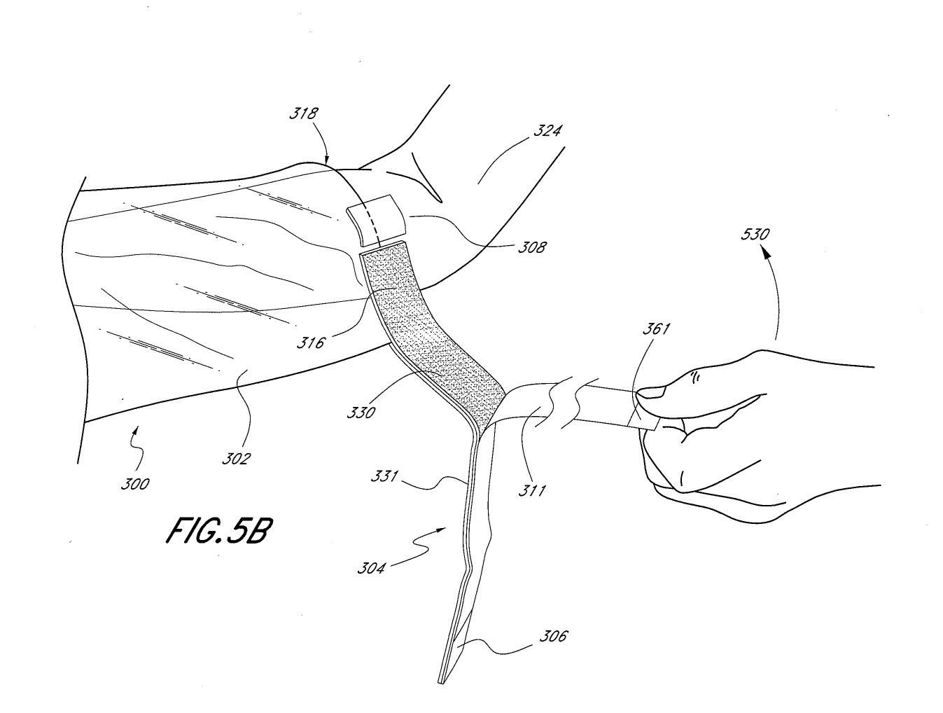

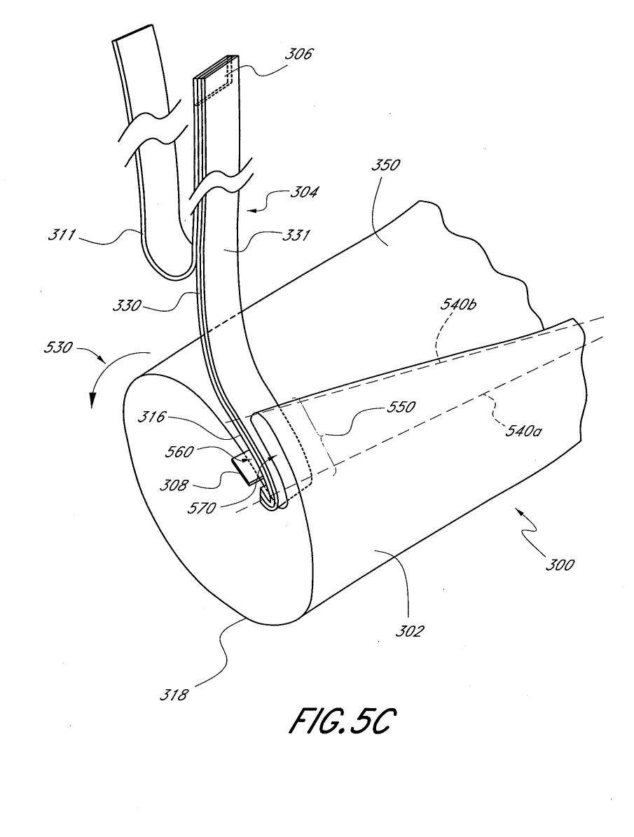

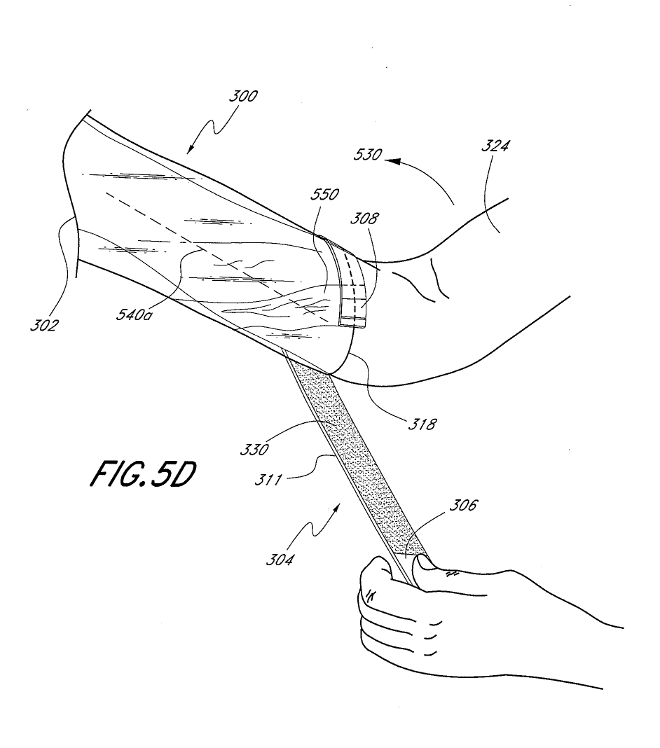

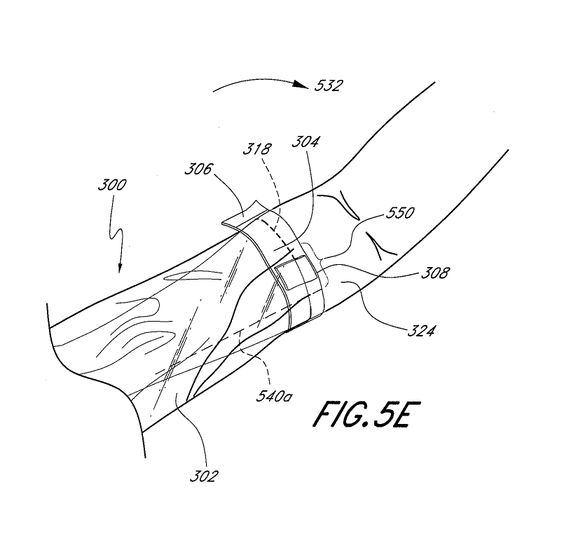

FIGS. 5A-5E show an exemplary method of using an embodiment of the medical barrier 300 shown in FIGS. 3A-4B. The user first inserts a portion of an appendage into the opening of the sheet 302 such that it is received and at least partially surrounded by the sheet 302, as described further herein. Next, the user severs the stabilization tab 308 from band 304, and removes at least a portion of the release layer 310 from the stabilization tab 308 as described further herein, and shown in FIGS. 4A-4B. As shown in FIG. 5A, the user applies the exposed portion 322 (FIG. 4B) of the adhesive layer of the stabilization portion 308, adhesive side-down, overlapping a portion of both the edge 318 of the sheet 302 and the user's skin (e.g., of appendage 324). In some embodiments, the step of applying the adhesive layer 322 of the stabilization portion 308 to the sheet 302 and/or the user's skin comprises removing a portion of the release layer 310 (e.g., portion 310a shown in FIG. 4B), applying a portion of the adhesive layer 322 to the edge 318 of the sheet 302 and/or the user's skin, removing the remaining portion of the release layer 310 (e.g., portion 310b shown in FIG. 4B), and applying the remainder of the adhesive layer 322 to the sheet 302 and the user's skin. The stabilization portion 308 can thus hold the medical barrier 300 in place against the user's skin so as to prevent that portion of the edge 318 of the sheet 302 from rotating during the remaining steps of applying the medical barrier 300. The stabilization portion 308 also allows the user to first secure the medical barrier in place against the user's skin using a single hand, and then perform the remainder of the application steps of medical barrier 300 with the same hand.

As shown in FIG. 5B, the user next grasps the removal tab 361 and moves tab 361 away from the band 304 to peel away the release liner 311 and expose the remainder of the adhesive portion 330 of the band 304. During this step, prior to wrapping the band 304 around the user's appendage, the exposed portion of adhesive layer 330 faces radially outwardly, or away from sheet 302. This step can be performed while pulling the band 304 toward the stabilization tab 308 in the direction shown by the arrow 530, to wrap the band 304 around the edge 318 of the sheet 302 and the user's appendage 324.

Referring to FIGS. 5B-5D, the user continues to peel away the release liner 311 while wrapping the band 304 along the open top perimeter 318 of the medical barrier 300 to seal a portion of the barrier 300 to the user's appendage 324. Note that in FIG. 5C, the user's appendage is not shown for clarity. Also, FIG. 5C is actually a different embodiment with the band on an opposite end of a barrier sheet 302. As the user wraps the band 304 around the user's appendage in the direction shown by arrow 530, the exposed portion of adhesive layer 330 is moved from an outwardly facing position, to a radially inwardly facing position, towards the edge 318 of sheet 302. The seal between the edge 318 of sheet 302 and the user's appendage 324 is created through the combination of pressure and adhesion exerted against the medical barrier 300 by the band 304 and the manner in which the band 304 overlaps the sheet 302 and the user's appendage 324. In some embodiments, the user may grasp removal tab 361 and completely remove the release liner 311 to expose the adhesive portion 330 of the band 304 (FIG. 5D). The user can then grasp the handling tab 306, pull the band 304 toward the stabilization tab 308 in the direction shown by the arrow 530 and wrap the band 304 around the edge 318 overlapping the sheet 302 and the user's appendage 324. The seal between the top edge 318 of the medical barrier and the user's appendage 324 in this embodiment is created through the combination of pressure and adhesion exerted against the medical barrier 300 by the band 304 and the manner in which the adhesive layer 330 of band 304 straddles or overlaps both the sheet 302 and the user's appendage 324.

Referring to FIGS. 5C and 5D, stabilization tab 308 is placed so that the edge 318 of sheet 302 is held in place, and kept from moving relative to the user's appendage 324 as force as applied to the band 304 in the direction shown by the arrow 530. The sheet 302 can be held taut near the intersection between the stabilization tab 308 and the sheet 302. The band 304 is moved in the direction shown by arrow 530, using stabilization tab 308 as a fulcrum, forming an overlapping or “double-fold” section 550 of the excess perimeter material of sheet 302. Overlapping section 550 is formed along a sharp fold edge 540a near the intersection between the stabilization tab 308 and the top edge 318 of the sheet 302, and a sharp fold edge 540b near a distal edge of the attached end segment 316. In a preferred embodiment, fold edge 540a is formed against the edge of the stabilization tab 308. In a preferred embodiment, fold edge 540b is formed against the distal edge of the attached end segment 316. Using the stabilization tab 308 to form the fold edges 540a and 540b facilitates control of the excess perimeter material of sheet 302, allowing an inner fold layer 560 and an outer fold layer 570 of overlapping section 550 to be formed. Inner fold layer 560 is sealed by moving the portion of adhesive layer 330 extending along the attached end segment 316 of the band from a radially outward-facing position (FIG. 5B) to a radially inward-facing position (FIG. 5C) during the application of band 304. Using the stabilization tab 308 to form and seal fold edge 540a helps prevent sheet 302 from bunching and forming a series of pleats. This configuration helps ensure that the adhesive layer 330 of band 304 securely seals inner fold layer 560 under an outer fold layer 570 of the overlapping section 550 to prevent leaks due to unwanted channels within the barrier 300. The stabilization tab 308 and the fold edge 540a can be placed so that they can be easily seen by the user.

The use of the stabilization tab 308 allows a greater wrapping force, or torque, to be used during the application of band 304 while forming overlapping section 550. Specifically, when the user wraps the band 304 around the edge 318, torque that would otherwise cause the sheet 302 to spin or slip relative to the appendage is preferably absorbed at a folding point created by stabilization tab 308 (i.e., the fulcrum point formed at the interface between fold edge 540a and the edge of the stabilization tab 308). The stabilization tab 308 helps isolate a portion of the fold in sheet 302, and helps generate a substantially straighter fold extending generally along the longitudinal axis of the appendage. A greater wrapping force can also help ensure that the fold edge 540a has a tighter, substantially straighter edge. A tighter, substantially straighter-edged fold edge 540a is less prone to channel formation than a series of loose folds or pleats around a medical barrier.

FIG. 5D shows the band 304 with the remainder of the release liner 311 removed, exposing the remainder of adhesive layer 330. FIG. 5D shows the band 304 being further wrapped around the user's appendage 324 in the direction 530 by a user grasping the handling tab 306.

FIG. 5E shows the band 304 with the remainder of the adhesive layer 330 applied over the edge 318 of the sheet 302 around the remainder of the perimeter of the user's appendage to form a continuous seal around the perimeter of the appendage between the edge of the sheet and the patient's skin. As described further herein, in some embodiments, the perimeter of the edge 318 of sheet 302, and the length of the adhesive layer 330 of band 304 will typically exceed the length of the perimeter of the user's appendage in the area of coverage. In some embodiments, the length of the band 304 will sufficiently exceed the length of the perimeter of the user's appendage to allow the adhesive layer 330 of band 304 to seal the inner fold layer 560 (FIG. 5C) and to further cover and seal the outer fold layer 570 and an outer portion of edge 318 extending along the overlapping portion 550 (FIGS. 5C and 5E). Attaching a portion (e.g., portion 316) of the band 304 to the outer surface 350 of the sheet 302 and moving the band so that the adhesive layer 330 faces inwardly while being wrapped around the appendage can assure that the entire circumference of the sheet's edge 318 is covered by adhesive during application, including the area under the overlapping section 550 (e.g., inner fold layer 560 and outer fold layer 570). This continuous adhesive seal around the entire circumference of the sheet 302 helps to prevent any ingress of fluids into the protected area that might otherwise occur due to unwanted fold formations or channels.

Embodiments may differ as to the length of engagement between band 304 and sheet 302 (e.g., the length of attachment portion 316), which in turn affects the size of the overlapping section 550 (FIGS. 5C-5E). The length of attachment portion 316 can be expressed as a percentage of the length (e.g. circumference) of opening 318a (FIG. 3A). In some embodiments, attachment portion 316 can have a length that can range from approximately 15% to 75%, or more narrowly, from approximately 25% to 50%, or even more narrowly, from approximately 35% to 40% of the circumference of opening 318a.

After the medical barrier 300 has been applied to a user's appendage 324, as shown in FIG. 5E, and needs to be removed or replaced, the user may grasp handling tab 306 and apply force in the direction shown by the arrow 532, to remove the band 304 from the user's appendage 324.

In some embodiments, one or more adhesive strips (not shown), separate from the band, can be disposed to extend partially along a length of the edge of the sheet, and on the inner and/or outer surface of the sheet. In an embodiment, an adhesive strip extends partially along a length of the edge of the sheet and on the outer surface of the sheet, facing outwardly from the sheet. In such embodiment, the adhesive strip can be used to seal the inner fold layer as the sheet is wrapped around a user's appendage and as the inner fold layer is folded onto the adhesive strip. In another embodiment, an adhesive strip extends partially along a length of the edge of the sheet and on the inner surface of the sheet, facing inwardly from the sheet. In such embodiment, the adhesive strip can be used to seal the outer fold layer as the sheet is wrapped around a user's appendage and as the outer fold layer is folded onto the adhesive strip. These adhesive strips can be provided instead of or in addition to the outwardly-facing adhesive layer on the band, or can be provided with a band with an inwardly-facing adhesive layer, and can provide a similar channel-preventing function within the inner and/or outer fold layers.

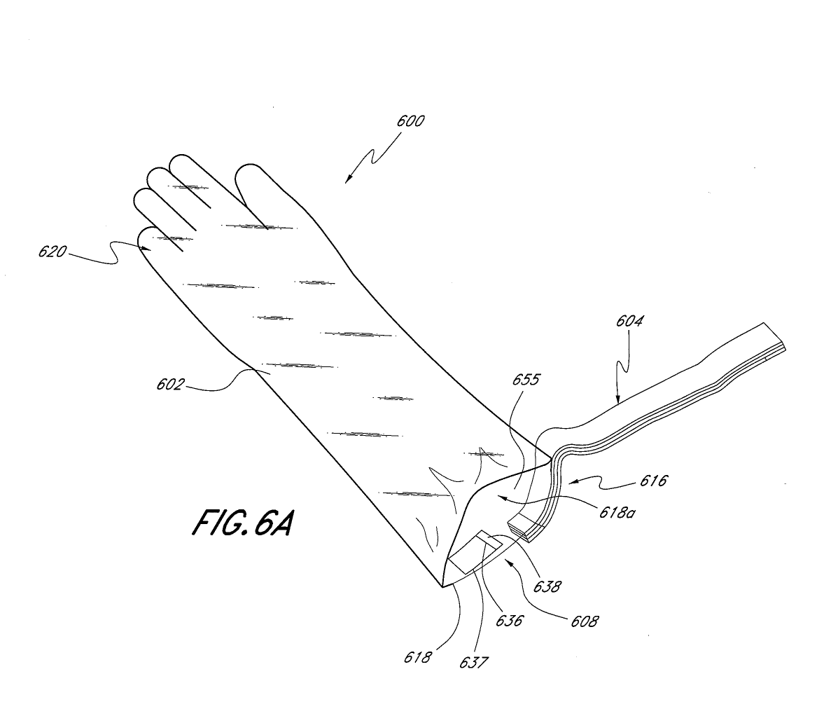

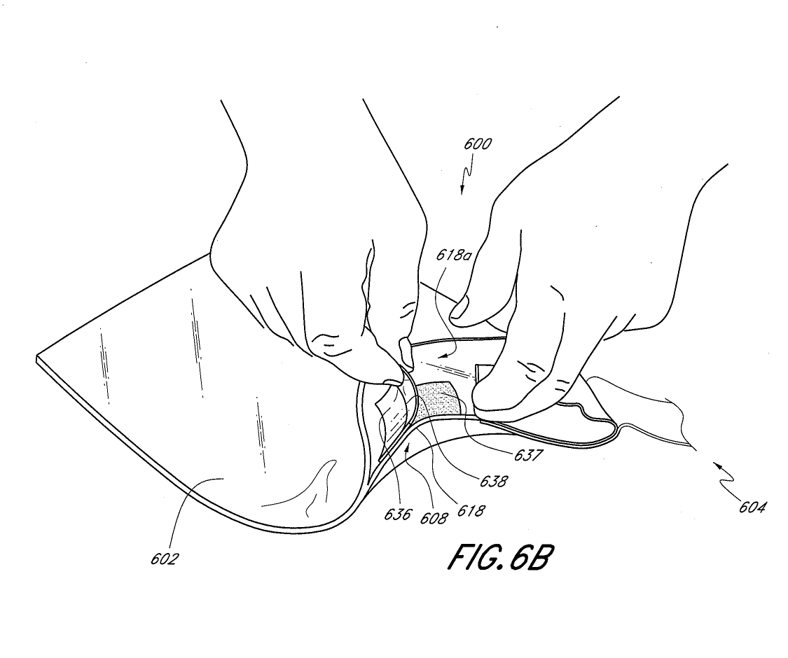

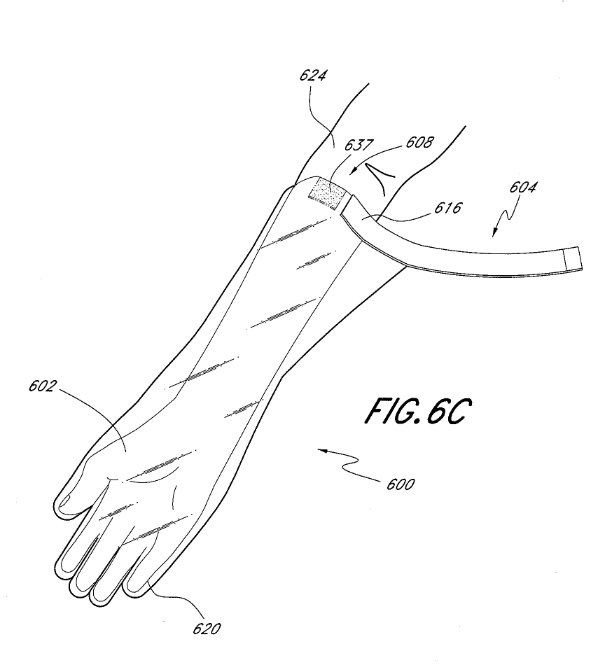

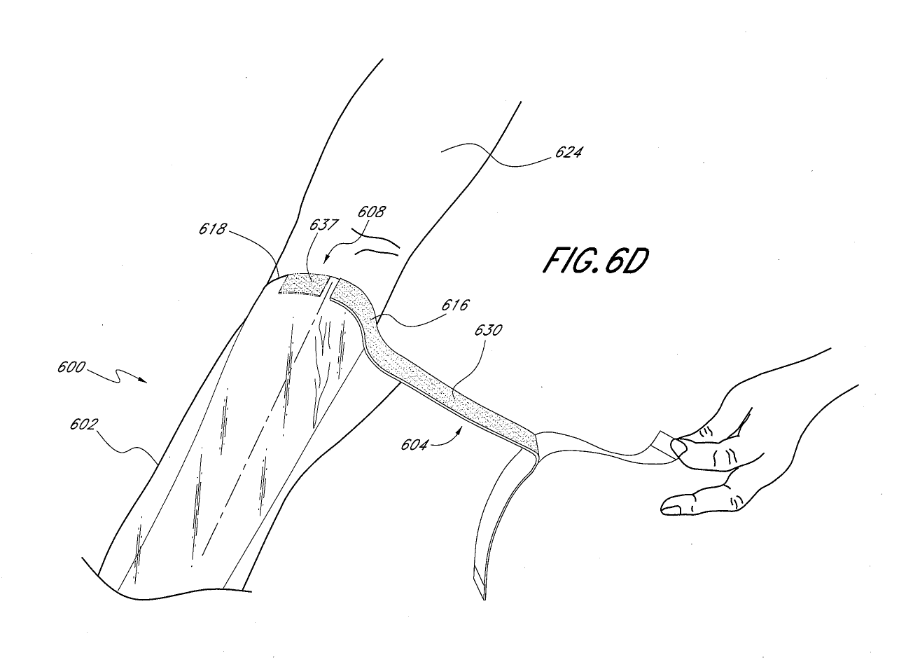

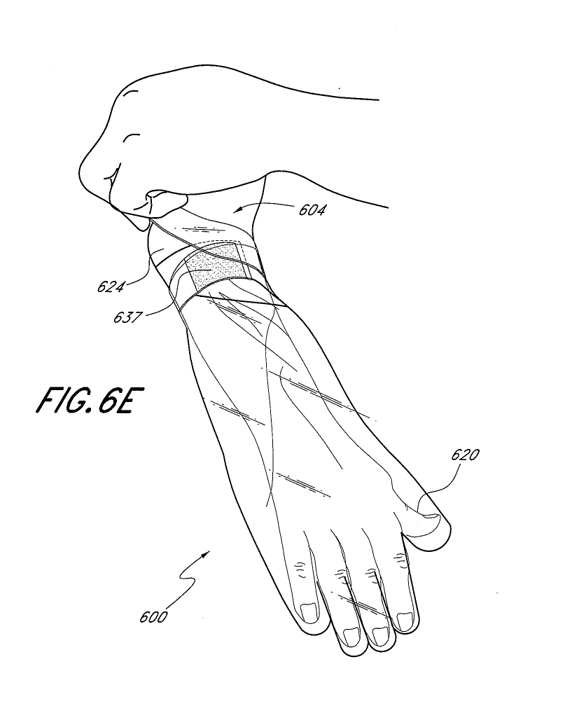

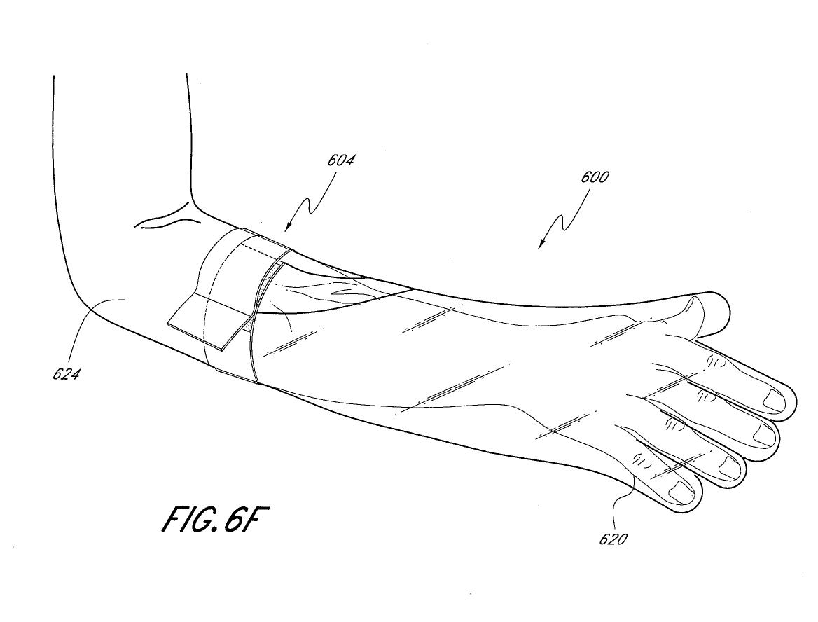

FIGS. 6A-6F show another embodiment of and methods for using a medical barrier 600. Many of the components in the embodiment of medical barrier 600 are substantially similar to the components of the embodiments of medical barriers 100 and 300 shown in FIGS. 2-5E, and function similarly to the manner described herein for medical barriers 100 and 300. Medical barrier 600 can comprise a sheet 602 with an edge 618 circumscribing an opening 618a at one end. Sheet 602 is shown, for exemplary purposes only, with a single band 604 proximate to the opening 618a, and with a glove-shaped closed end 620. Band 604 is shown, for exemplary purposes only, with a connected portion 616 connected to the sheet 602 and an adhesive layer 630. One difference between the medical barrier 600 and the medical barriers 100 and 300 is that the medical barrier 600 can comprise an inner stabilization tab 608 on an inner surface 655 of sheet 602. Inner stabilization tab 608 can comprise an inner adhesive layer 637 extending at least partially around a portion of the perimeter of the edge 618, inside opening 618a, along the inner surface 655 of sheet 602. Inner stabilization tab 608 can further comprise an inner release liner 636, with a removal tab 638 to more easily allow inner release liner 636 to be peeled from inner adhesive layer 637, to expose the adhesive thereon.

FIGS. 6B-6F show a method of applying the medical barrier 600 to a portion of a user's appendage 624. The steps used in applying the medical barrier 600 to a user's appendage 624 are substantially similar to those described herein for medical barriers 100 and 300, and those shown in FIGS. 5A-5E. One difference in applying the illustrated medical barrier 600 is the method of applying the inner stabilization tab 608, as shown in FIGS. 6B and 6C, and described presently.

FIG. 6B shows a user preparing to apply the medical barrier 600 using the inner stabilization tab 608. The inner stabilization tab 608 functions substantially similar to the other stabilization tabs described herein. Specifically, when exposed and adhered to a user's appendage, tab 608 can prevent the medical barrier 600 from twisting or spinning as the user applies sheet 602 with band 604. However, applying inner stabilization tab 608 differs from the other stabilization tabs described herein, in that in use, the user grasps tab 638 and peels the inner release layer 636 away from the inner adhesive layer 637, thereby exposing the adhesive layer 637. The user subsequently places the barrier 600 over a portion of a user's appendage and applies the edge 618 to the user and adhesive 637, as shown in FIG. 6C.

Once the adhesive 637 of the inner stabilization tab 608 is applied, the medical barrier 600 can be applied to the user's appendage 624, as shown in FIGS. 6D-6F. The method of applying the medical barrier 600 shown in FIGS. 6D-6F is substantially similar to the method of applying the medical barrier 300 described above and shown in FIGS. 5B-5E. Another optional difference in the illustrated method of applying the medical barrier 600 shown in FIGS. 6D-6F is that, for exemplary purposes, the connected portion 616 of band 604 does not overhang the edge 618. Thus, to form a continuous seal around the perimeter of sheet 602 between the edge 618 of the sheet 602 and the user's skin, the remainder of the band 604 can be angled slightly relative to the edge 618 as the band 604 is wrapped around the sheet 602. The band 604 is wrapped around the user's appendage and sheet 602 such that the adhesive layer 630 overhangs the edge 618 and is applied to both a portion of the user's skin and the edge 618 of sheet 602. In some embodiments, the band 604 of medical barrier 600 can be wrapped around the sheet 602 without contacting the user's skin, rather than overlapping the edge 618 of opening 618a. In such embodiments, a continuous pressure seal can be formed around the edge 618 of sheet 602 between the user's skin and the inner surface of sheet 602, instead of the band 604 forming a continuous adhesive seal across the user's skin and the edge 318 of sheet 302, as shown in FIGS. 5B-5E and FIGS. 6D-6F and described above. It will be understood that the various embodiments of the medical barriers described herein can have bands that form a pressure seal without contacting the user's skin or, alternatively, bands that form both a pressure seal and an adhesive seal across the edge.

FIGS. 7A and 7B show perspective and partially exploded views of a medical barrier 700 in accordance with another embodiment. Many of the components in the medical barrier 700 are substantially similar to the components of the medical barriers 100, 300 and 600 shown in FIGS. 2-6F, and function similarly to the manner described above for medical barriers 100, 300 and 600. Medical barrier 700 can comprise a sheet 702 with an edge 718 circumscribing an opening 718a at one end. Sheet 702 is shown, for exemplary purposes only, as a generic, waterproof or water resistant bag with a single band 704 proximate to the opening 718a, but can be shaped according to the application at hand. The band 704 can comprise an attachment portion, e.g., end segment 716 attached to an outer surface 750 of the end of the sheet 702, the segment 716 oriented generally along a portion of the perimeter, e.g., circumference, of the edge 718. The band 704 can comprise a proximal end portion 761 of a release layer 711 that is substantially similar to and functions similarly to the proximal end portion 361 of release layer 311 for medical barrier 300 (FIGS. 3A-3B). The main difference with the proximal end portion 761 is that in some embodiments, it is a substantially smaller width than band 704. In a preferred embodiment, proximal end portion 761 extends at an angle relative to band 704. In a further preferred embodiment, proximal end portion 761 extends at an approximately transverse angle relative to band 704.

An overhanging portion 770 of a width of the attached end segment 716 can extend beyond the edge 718 of the sheet 702. The overhanging portion 770 can comprise a portion of release layer 711, adhesive layer 730, and/or substrate layer 731, extending at least partially along the length of attached segment 716. In FIGS. 7A-7B, the layers 711, 730, and 731 are shown separated from one another for clarity. In some embodiments, one or more of release layer 711, adhesive layer 730, and/or substrate layer 731 do not extend beyond the edge 718 of the sheet 702 along the entire length of attached segment 716. One difference between the illustrated medical barrier 700 with the medical barriers 100, 300 and 600 is that the overhanging portion 770 of the attached end segment 716 can comprise a removable stabilization tab portion, either of the substrate 731 (FIG. 7A), or the release layer 711 (FIG. 7B), predisposed to be peelable from the adhesive layer 730, and severable from a remainder of the band 704, as will be described presently.

Referring to FIG. 7A, in an embodiment of medical barrier 700, the attached end segment 716 can comprise a removable stabilization tab substrate portion 775 of the substrate 711. The removable stabilization tab substrate portion 775 can be severed or predisposed to be severable from the remainder of the substrate 731, using any of the methods described herein (e.g., aligned perforations or a kiss cut) or otherwise known in the art. The stabilization tab substrate portion 775 can be materially the same or, alternatively, materially different than the remainder of substrate 731, or can be coated the same or, alternatively different as substrate portion 775. Stabilization tab substrate portion 775 can comprise the same material as release layer 711. Stabilization tab substrate portion 775 can comprise a tab handling portion 762 extending beyond the edge of overhang portion 770, to facilitate removal of tab 775. When stabilization tab substrate portion 775 is removed, an inwardly-facing underlying stabilization tab adhesive portion 773 of the adhesive layer 730 is exposed. The stabilization tab substrate portion 775, the stabilization tab adhesive portion 773, and the release layer 711 can comprise materials with properties that allow stabilization tab substrate portion 775 to be removed without removing the stabilization tab adhesive portion 773 from release layer 711. When exposed, stabilization tab adhesive portion 773 can be applied to a user, to secure and stabilize the edge 718 during the application of sheet 702, and thus function similarly as the other stabilization tabs described herein. The stabilization tab adhesive portion 773 can comprise a material with sufficient strength and adhesive properties to secure and stabilize the edge 718 during the application of sheet 702 to the user's skin.

In an embodiment, a slit 740a extends at least partially across the width of stabilization tab substrate portion 775. In a preferred embodiment, the slit 740a extends approximately orthogonally with respect to the edge 718. The slit 740a can be formed prior to band 704 being attached to sheet 702 or after band 704 is attached to sheet 702. In some embodiments, the slit 740a resembles a gap. In some embodiments, the slit 740a resembles a thin cut. The slit 740a can be shaped like a line, rectangular gap, v-gap, or curved slit and the like. There may also be multiple slits along the band 704. Slit 740a in conjunction with stabilization tab adhesive portion 773 provides a fold point during the application of sheet 702, to form a sharp fold edge 740, which functions similarly to the sharp fold edge 540a described above and shown in FIGS. 5A-5E.

FIG. 7B shows an embodiment of medical barrier 700 in which the attached end segment 716 comprises a removable stabilization tab release layer portion 778 of the substrate 711. The removable stabilization tab release layer portion 778 can be severed or predisposed to be severable from the remainder of the release layer 711, using any of the methods described herein (e.g., aligned perforations or a kiss cut) or otherwise known in the art. The stabilization tab release layer portion 778 can be peelable from the adhesive layer 730 without removing a remainder of the release layer 711. The stabilization tab release layer portion 778 can comprise a portion 762 extending beyond the edge of overhang portion 770, to facilitate removal of tab 778. When the stabilization tab release layer portion 778 is removed, an outwardly-facing underlying stabilization tab adhesive portion 773 of the adhesive layer 730 is exposed. Stabilization tab adhesive portion 773 can be moved to an inwardly-facing position by folding portion 773 under the edge 718 of opening 718a, in the direction shown by arrow 779. The stabilization tab adhesive portion 773 can be applied to a user, to secure and stabilize the edge 718 during the application of sheet 702, and thus function similarly as the other stabilization tabs described herein. It can be advantageous to fold stabilization tab adhesive portion 773 under the edge 718 of opening 718a, so that the attachment point of the tab 773 to the user is aligned, e.g., substantially centered, relative to the pulling force by the user on band 704 during the application of sheet 702. In a preferred embodiment, a slit 740b extends partially across the width of overhang portion 770, through one or more of the overhanging portions of release layer 711, adhesive layer 730, and/or substrate 731. The slit 740b can be substantially similar to and function similarly to slit 740a described above and shown in FIG. 7A. In a preferred embodiment, the slit 740b can differ from slit 740a by extending through each of the overhanging portions of release layer 711, adhesive layer 730, and substrate 731. In a further preferred embodiment, the slit 740b extends approximately orthogonally with respect to the edge 718.

During use, the user inserts an appendage (not shown for clarity) into the sheet 702, as described in the other medical barrier embodiments herein. Next, the user removes the removable stabilization tab substrate portion 775 (FIG. 7A) or the removable stabilization tab release layer portion 778 (FIG. 7B) from the remainder of the substrate 731 or release layer 711, respectively, to expose the stabilization tab adhesive layer portion 773. The user applies the exposed stabilization tab adhesive layer portion 773 to a portion of the patient's skin. This will secure and stabilize a portion of the sheet to the patient's skin so as to prevent that portion of the sheet from rotating during application of the medical barrier, as described in the other stabilization tab embodiments described herein. In order to apply the exposed stabilization tab adhesive layer portion 773 of the embodiment shown in FIG. 7B, the user will first need to fold the stabilization tab adhesive layer portion 773 under the edge 718 of opening 718a (optionally before removing the stabilization tab release layer portion 778). The user grasps and pulls the proximal end portion 761 to remove the release layer 711 (or, in the embodiment of FIG. 7B, the remainder of the release layer 711) from a remainder of the band 704 to expose the adhesive layer 730 (or, in the embodiment of FIG. 7B, the remainder of the adhesive layer 730). Next, the user applies the exposed adhesive layer 730 of the band 704 to the sheet 702 using the steps described herein for the various embodiments herein, e.g., for medical barrier 300, as shown in FIGS. 5B-5E.

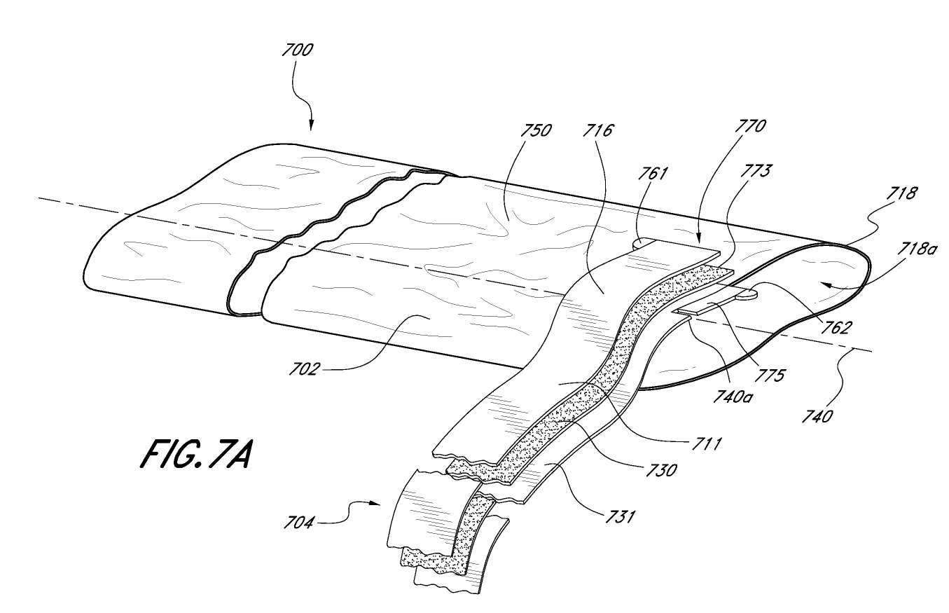

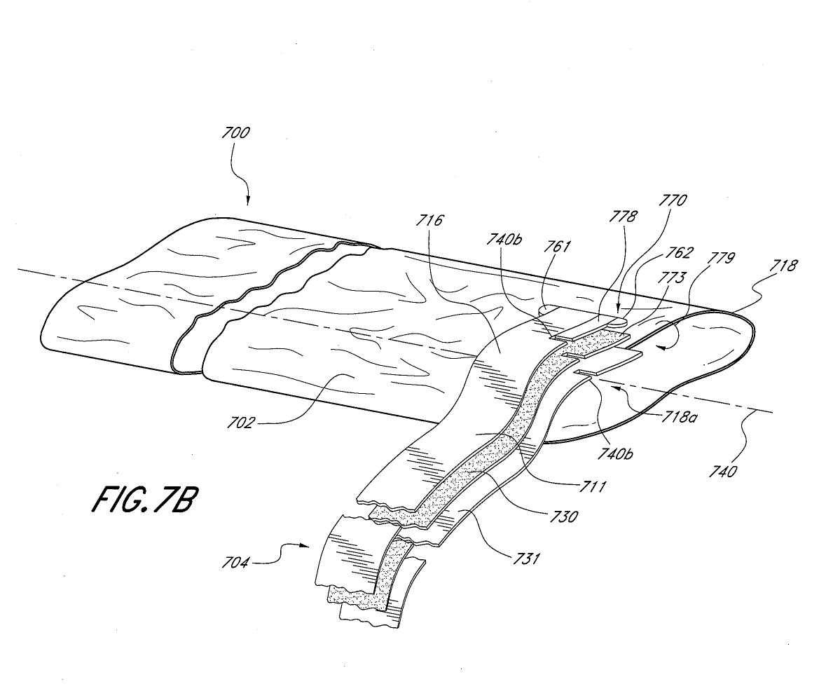

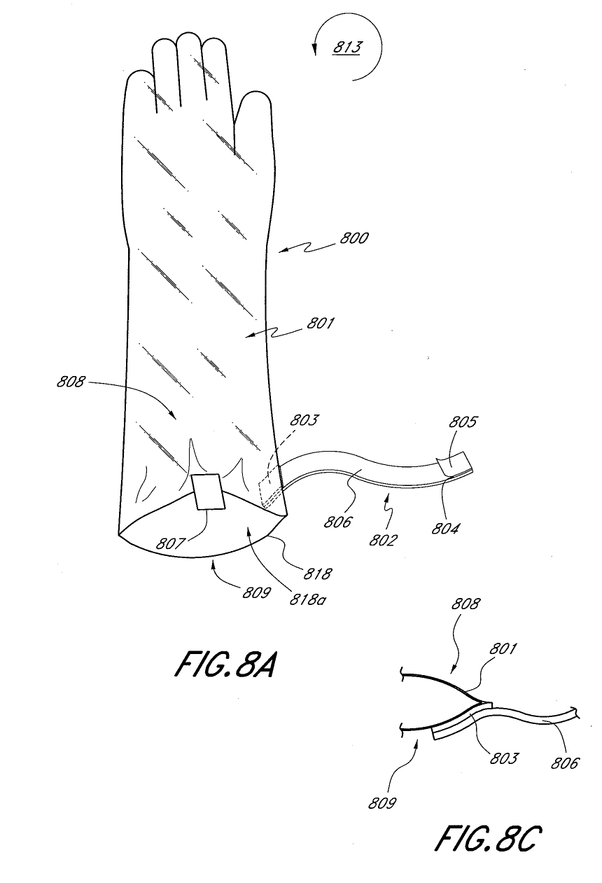

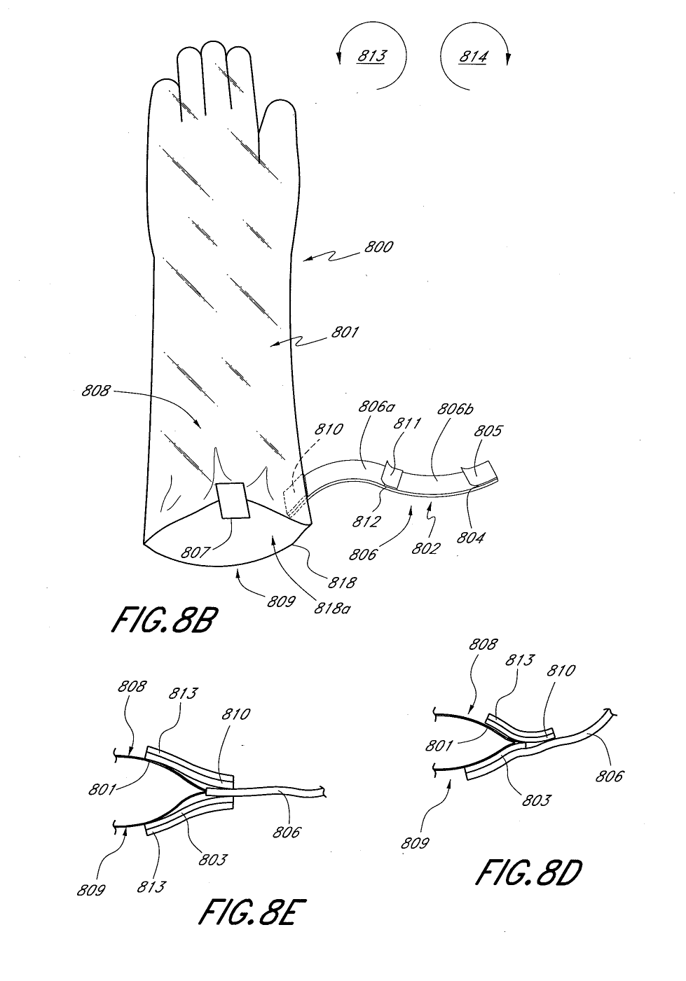

FIGS. 8A and 8B show embodiments of a medical barrier 800 comprising a glove-shaped sheet 801, an elastic band assembly 802 and a stabilization tab 807. Many of the components in the illustrated medical barriers 800 are substantially similar to and function similarly to the manner of the components of the other embodiments of medical barriers described herein. For example, the illustrated embodiments show the sheet 801 with an edge 818 circumscribing an opening 818a at one end of the sheet 801 for exemplary purposes only; medical barrier 800 can comprise more than one opening. In the illustrated embodiments, the stabilization tab 807 is shown overhanging the edge 818, on an outer surface of the sheet 801, although tab 807 can alternatively be positioned on the inner surface of sheet 801, or severably connected to band assembly 802, as described in the other embodiments herein. The sheet 801 is not limited to a glove shape and may take on various forms described herein and known in the art, such as a foot shape, tube shape or bag-like shape.

The elastic band assembly 802 can comprise an elastic band 806, an adhesive strip 803 connecting the band 806 to sheet 801, and an adhesive region 804 disposed along a length of one side of the band 806 to secure the band 806 around a user's appendage during use. A peelable, or removable tab 805 can be configured to cover and protect the adhesive region 804, and can comprise similar materials and function similarly to the adhesive release layers disclosed further herein. The adhesive region 804 can be disposed along the length of one side of the band 806 in many different configurations. In the embodiment of FIG. 8A, a single adhesive region 804 is disposed proximate to a free end of elastic band 806. Referring to FIG. 8B, in some embodiments, a plurality of adhesive regions 804, 812 with respective peelable tabs 805, 811 can be disposed along elastic band 806, dividing the elastic band 806 into a plurality of segments. In the embodiment shown in FIG. 8B, a first segment 806a of elastic band 806 extends between its proximal end (attached to sheet 801) and adhesive region 812, and a second segment 806b of elastic band 806 extends between adhesive region 812 and adhesive region 804. In some embodiments, three or more segments can divide elastic band 806. Referring to both FIGS. 8A and 8B, a proximal end portion of the elastic band 806 can be attached, or connected, at or near a bottom side 809 of the sheet 801 with the adhesive strip 803 (see also FIG. 8C). The elastic band 806 can comprise similar materials and function similarly to the other flexible bands described herein. In a preferred embodiment, the elastic band 806 can comprise various materials known to the industry that can provide adequate elasticity to compress the sheet 801 against the user's appendage when elastic band 806 is stretched and wrapped around such appendage, such as natural or synthetic rubber materials. In a further preferred embodiment, the elastic band 806 comprises polyisoprene.

Referring to FIG. 8A, in use (according to certain embodiments), the user slides an appendage (not shown for clarity) into the opening 818a of sheet 801 and attaches the edge 818 of the sheet 801 to the appendage using an exposed adhesive strip on the surface of the stabilization tab 807. The user removes the peelable tab 805, exposing the adhesive region 804 on band 806. The user stretches and wraps the elastic band assembly 802 in the direction 813 circumscribing the appendage, thereby compressing a portion of the sheet 801 at or near the edge 818 against the appendage and forming a seal between the sheet 801 and the user's skin. The adhesive region 804 is then secured to the sheet 801 and/or the user's skin, so that the elastic band 806 creates a compression seal between the external environment and the interior of sheet 801. In some embodiments, the peelable tab 805 can be removed from the adhesive region 804 after wrapping the elastic band assembly 802 around the appendage, and prior to securing the adhesive region 804 to the sheet 801 and/or the user's skin.

Referring to FIG. 8B, in use (according to certain embodiments), the user slides an appendage (not shown for clarity) into the opening 818a of sheet 801 and attaches the edge 818 of the sheet 801 to the appendage using an exposed adhesive strip on the surface of the stabilization tab 807. The user removes the peelable tab 811, exposing the adhesive region 812 on band 806. The user grasps the elastic band assembly 802 (in a preferred embodiment, proximate to adhesive region 812) and applies a first pulling force to the first segment 806a of elastic band assembly 802, thereby stretching the first segment 806a of the elastic band 806. The user wraps the first segment 806a of the elastic band 806 in the direction 813, to at least partially circumscribe the appendage and the sheet 801, thereby compressing at least a partial section of the sheet 801 against the appendage and forming a seal between the sheet 801 and the user's skin. The adhesive region 812 can then be secured to the sheet 801 and/or the user's skin, with the first segment 806a of the elastic band 806 thereby creating a compression seal between the external environment and the interior of at least a partial section of the sheet 801. In some embodiments, the peelable tab 811 can be removed from the adhesive region 812 after wrapping the elastic band assembly 802 around the appendage, and prior to securing the adhesive region 812 to the sheet 801 and/or the user's skin Next, the user removes the peelable tab 805, exposing the adhesive region 804. The user grasps the remainder of the elastic band assembly 802 and applies a second pulling force to stretch the second segment 806b of elastic band assembly 802. The user wraps the second segment 806b of the elastic band 806 in the direction 813, to at least partially circumscribe the appendage and a remaining perimeter section of the sheet 801, thereby compressing the remainder of the sheet 801 against the appendage and forming a seal between the sheet and the user's skin. The adhesive region 804 can then be secured to the sheet 801 and/or the user's skin, with the elastic band 806 thereby creating a compression seal between the external environment and the interior of at least a partial section of the sheet 801. In some embodiments, the peelable tab 805 can be removed from the adhesive region 804 after wrapping the elastic band assembly 802 around the appendage, and prior to securing the adhesive region 804 to the sheet 801 and/or the user's skin.

An advantage of providing a plurality of adhesive regions along elastic band assembly 802 is that sheet 801 can be applied with varying tensions around its circumference, for an improved fit to the appendage. Thus, in the embodiment of FIG. 8B, the tension in the first band segment can be different than the tension in the second band segment. Another advantage of providing a plurality of adhesive regions along elastic band assembly 802 is that the elastic band 806 can be wrapped around the perimeter of the user's appendage in one or more steps, which makes it easier for the user to apply the medical barrier 800 with a single hand.

FIGS. 8C-8E show close-up, partial side views of various embodiments of the elastic band assembly 806 connected to sheet 801 shown in FIGS. 8A and 8B. In FIG. 8C, an end portion of the elastic band 806 can be attached, or connected, to a bottom side 809 of the sheet 801 with the adhesive strip 803, wherein one side of the adhesive strip 803 is connected to the sheet 809, and a second side of the adhesive strip 803 is connected to the end portion of the elastic band 806, as described above.

In FIGS. 8D and 8E, the medical barrier 800 can comprise a first adhesive strip 803 to connect an end portion of the band 806 to the sheet, and a second adhesive strip 810 to connect the end portion of the band 806 to the sheet 801.

Referring to the embodiment shown in FIG. 8D, the first adhesive strip 803 can comprise a first side adhered to one side of the end portion of the band 806, and a second side adhered to the sheet 801. In a preferred embodiment, the first adhesive strip is adhered to the bottom side 809 of the sheet 801. A first portion of a first side of the second adhesive strip 810 can be adhered to the same side of the end portion of the band 806, and a second portion of the first side of the second adhesive strip 810 can be adhered to the sheet 801. In a preferred embodiment, the second portion of the first side of the second adhesive strip 810 is adhered to a top side 808 of the sheet 801. A second side of the adhesive strip 810 can be covered with a release layer section or substrate section 813, functioning similarly to the other release layers or substrate layers described further herein.

Referring to the embodiment shown in FIG. 8E, the end portion of the band 806 can comprise a first side and an opposite second side. A first portion of a first side of the first adhesive strip 803 can be adhered to the first side of the end portion of the band 806. A second portion of the first side of the first adhesive strip 803 can be adhered to the sheet 801. In a preferred embodiment, the second portion of the first side of the first adhesive strip 803 can be adhered to the bottom side 809 of the sheet 801. A second side of the adhesive strip 803 can be covered with a release layer section or substrate section 813, functioning similarly to the other release layers or substrate layers described further herein.

A first portion of a first side of the second adhesive strip 810 can be adhered to the second side of the end portion of the band 806. A second portion of the first side of the second adhesive strip 810 can be adhered to the sheet 801. In a preferred embodiment, the second portion of the first side of the second adhesive strip 810 can be adhered to the top side 808 of the sheet 801. A second side of the adhesive strip 810 can be covered with a release layer section or substrate section 813, functioning similarly to the other release layers or substrate layers described further herein. An advantage of using a second adhesive strip 810 to connect the end portion of the band 806 to sheet 801 is that strips 810 and 803 reinforce the attachment of band 806 to sheet 801, on both sides of sheet 801, allowing a user to apply greater force to band assembly 802, regardless of the direction of the force (e.g., in either directions shown by direction arrows 813 and 814 in FIG. 8B). Allowing a greater force to be applied to band assembly 802 will increase the strength of its pressure seal, and prevent leakage.

Various components of the embodiments of the medical barriers described herein may be labeled to instruct a user on how and in what order to apply or remove the various components. For example, without limitation, the components may be color-coded, numbered, and/or otherwise labeled such that it is easy for user to determine the order in which to remove, wrap, and/or apply any or all of the stabilization tab(s), release layer(s), adhesive layer(s), band(s), and/or sleeve, or portions thereof, for convenient application of the medical barriers. In some embodiments, the tabs are labeled with detailed instructions for the user, e.g., describing how to remove the release layer and how to apply the underlying adhesive directly or indirectly to a patient's appendage. Through such labeling or other configuration, the presently disclosed medical barrier can guide users to remove the tabs in the proper order to obtain the most desirable application, sealing, and removal properties of the medical barriers.

Although certain preferred embodiments and examples have been discussed herein, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the present disclosure, including the appended claims.

What is claimed is:

1. A medical barrier comprising:
a sheet comprising an edge circumscribing an opening disposed at a first end of the sheet, through which a portion of a patient's appendage can be inserted so as to be at least partially surrounded by the sheet; and
a flexible band connected to the sheet, the band comprising:
a substrate;
an adhesive layer; and
a release layer disposed on the adhesive layer so that the adhesive layer is between the substrate and the release layer, the release layer being peelable from the adhesive layer to expose the adhesive layer;
wherein a portion of an end of the band comprises a stabilization tab that includes portions of the substrate, adhesive layer, and release layer, the stabilization tab predisposed to be severable from a remainder of the band.

2. The medical barrier of claim 1, wherein the band is perforated to permit severing of the stabilization tab from the remainder of the band.

3. The medical barrier of claim 1, further comprising a second opening disposed at a second end of the sheet, the second end being opposite the first end.

4. The medical barrier of claim 1, further comprising a closed second end of the sheet shaped to accommodate a patient's hand or foot, the second end being opposite the first end.

5. The medical barrier of claim 1, wherein the release layer comprises a main portion and a substantially smaller handling portion, the handling portion being at or near a free end of the band, the main portion and the handling portion being severed or predisposed to be severable with respect to each other, such that the main portion of the release layer can be removed from the band without removing the handling portion of the release layer.

6. The medical barrier of claim 1, wherein the substrate is attached to the sheet at or near a proximal end of the substrate, a proximal end portion of the release layer extending proximally beyond the proximal end of the substrate and beyond a proximal end of the adhesive layer, the proximal end portion of the release layer being detached from the sheet.

7. A method of applying the medical barrier of claim 1, comprising:
severing the stabilization tab from the remainder of the band;
removing the portion of the release layer from the stabilization tab to expose the adhesive layer portion of the tab;
inserting the patient's appendage into the opening so as to be at least partially surrounded by the sheet;
adhering a first portion of the exposed adhesive layer of the tab to the edge of the sheet;
adhering a second portion of the exposed adhesive layer of the tab to the patient's skin to limit movement of the barrier with respect to the patient's skin;
removing the release layer from the remainder of the band to expose a remainder of the adhesive layer; and
adhering the remainder of the adhesive layer along the edge of the sheet.

8. A medical barrier comprising:
a sheet comprising an edge circumscribing an opening disposed at an end of the sheet, through which a portion of a patient's appendage can be inserted so that the edge surrounds a perimeter of the appendage; and
a flexible band connected to the sheet, the band comprising:
a substrate;
an adhesive layer; and
a release layer having an inner surface disposed on the adhesive layer, so that the adhesive layer is between the substrate and the release layer, the release layer being peelable from the adhesive layer to expose the adhesive layer;
wherein an end segment of the band is attached to the sheet such that a portion of the release layer extending along the end segment of the band has an outer surface that faces away from the sheet, the end segment of the band oriented substantially along a circumference of the sheet's edge and positioned substantially at said edge.

9. The medical barrier of claim 8, wherein the end segment of the band that is attached to the sheet has a length of at least about 15-75% of the circumference of the sheet opening.

10. The medical barrier of claim 8, wherein at least a portion of the end segment of the band overhangs a portion of the edge of the sheet.

11. The medical barrier of claim 8, wherein the band is configured to form a continuous seal around the edge of the sheet between the sheet and the patient's appendage.

12. A medical barrier comprising:
a sheet comprising an edge circumscribing an opening disposed at an end of the sheet, through which a portion of a patient's appendage can be inserted so as to be at least partially surrounded by the sheet; and
a flexible band comprising:
a substrate;
an adhesive layer having an inner surface disposed on the substrate;
and
a release layer having an inner surface disposed on an outer surface of the adhesive layer so that the adhesive layer is between the substrate and the release layer, the release layer being peelable from the adhesive layer to expose the outer surface of the adhesive layer;
wherein a segment of the band is attached to an outer surface of the end of the sheet such that an outer surface of the release layer at the band's attached segment faces away from the sheet, the attached segment of the band being oriented generally along a circumference of the edge, an overhang portion of a width of the attached segment of the band extending beyond the edge;
wherein a removable stabilization tab portion of the substrate or the release layer within the overhang portion of the band is severed or predisposed to be severed from a remainder of the substrate or release layer and is peelable from the adhesive layer to expose a stabilization tab portion of the adhesive layer, the stabilization tab portion of the adhesive layer configured to adhere to a patient's appendage when inserted through the opening.

13. The medical barrier of claim 12, wherein the segment of the band is attached to the outer surface of the end of the sheet with a second adhesive layer configured between the substrate and the sheet.

14. The medical barrier of claim 12, wherein a section of the removable stabilization tab portion extends beyond an edge of the adhesive layer.

15. The medical barrier of claim 12, wherein the removable stabilization tab portion comprises a portion of the release layer and is peelable from the outer surface of the adhesive layer, the overhang portion including at least one slit extending at least partially through the width of the band, the at least one slit allowing a stabilization tab portion of the substrate and the stabilization tab portion of the adhesive layer to fold around the edge of the sheet.

16. The medical barrier of claim 15, wherein the slit extends approximately perpendicular to the edge of the sheet.

17. The medical barrier of claim 12, wherein the removable stabilization tab portion comprises a portion of the substrate and is peelable from the inner surface of the adhesive layer.

18. A medical barrier comprising:
a sheet comprising an edge circumscribing an opening disposed at an end of the sheet, through which a portion of a patient's appendage can be inserted so as to be at least partially surrounded by the sheet;
an elastic band connected to the sheet;
a plurality of adhesive regions disposed on and spaced along a length of one side of the band, each adhesive region being covered by a peelable tab;
a first adhesive strip connecting a proximal end portion of the band to the sheet; and
a second adhesive strip connecting the proximal end portion of the band to the sheet.

19. The medical barrier of claim 18, wherein:
the first adhesive strip has a first side adhered to one side of the end portion of the band;
the first adhesive strip has a second side adhered to the sheet;
a first portion of a side of the second adhesive strip is adhered to said side of the end portion of the band; and
a second portion of the side of the second adhesive strip is adhered to the sheet.

20. The medical barrier of claim 18, wherein:
the end portion of the band has a first side and an opposite second side;
a first portion of a side of the first adhesive strip is adhered to the first side of the end portion of the band;
a second portion of the side of the first adhesive strip is adhered to the sheet;
a first portion of a side of the second adhesive strip is adhered to the second side of the end portion of the band; and
a second portion of the side of the second adhesive strip is adhered to the sheet.

21. The medical barrier of claim 18, further comprising a stabilization tab attached to the end of the sheet, the stabilization tab comprising an adhesive strip configured to secure the end of the sheet to the patient's appendage.

22. The medical barrier of claim 18, wherein the stabilization tab at least partially overhangs the edge at the first end of the sheet.
